(19) United States
(12) Patent Application Publication
(10) Pub. No.: US 2010/0145285 A1
(43) Pub. Date: Jun. 10, 2010

(54) MULTI-LUMEN CATHETER CONFIGURATION

(75) Inventor: Christopher D. Bosel, Bloomington, IN (US)

Correspondence Address: BRINKS HOFER GILSON & LIONE/INDY/COOK;BRINKS HOFER GILSON & LIONE, CAPITAL CENTER, SUITE 1100, 201 NORTH ILLINOIS STREET, INDIANAPOLIS, IN, 46204-4220, US

(73) Assignee: Cook Critical Care, Incorporated, Bloomington, IN (US)

(21) Appl. No.: 12/330,638

(22) Filed: Dec. 9, 2008

(51) Int. Cl. A61M 25/00

(52) U.S. Cl. 604/264

(57) ABSTRACT

A multi-lumen catheter for use in the extracorporeal treatment of bodily fluids comprises a tubular catheter body having a first septum extending along its length, and having a withdrawal port and an infusion port. First and second withdrawal lumens are disposed on one side of the septum for transporting fluids withdrawn from a body vessel through the withdrawal port to an extracorporeal treatment unit, such as a dialyzer. An infusion lumen is disposed on the other side of the septum for infusion of treated fluids from the dialyzer through the infusion port into the vessel. The catheter body has a second septum extending along a length of the first septum. A proximal portion of the second septum separates the first and second withdrawal lumens, and a distal portion of the second septum extends distal to the withdrawal port. The distal portion tapers to a termination point along the length of the first septum length, for easing entry of the catheter into the vessel.

### BACKGROUND

1. Technical Field

The present application relates generally to a medical device, such as a catheter, for use in transporting fluids. More particularly, the application relates to a multi-lumen catheter for transporting a fluid from the body of a patient for extracorporeal treatment, and returning the treated fluid to the patient's body.

2. Background Information

Dual lumen catheters are commonly used for transporting a bodily fluid for treatment external of the patient's body, a process generally referred to in the medical field as “extracorporeal” treatment, and thereafter returning the treated fluid to the body. The fluid is withdrawn from the body through one of the lumens of the catheter, generally referred to as the withdrawal lumen. The fluid is subjected to a treatment process, and thereafter returned (or “infused”) to the body through the other lumen, generally referred to as the infusion lumen.

In many cases, the extracorporeal treatment is carried out as part of a hemodialysis procedure. During hemodialysis, blood is withdrawn from a blood vessel through the withdrawal lumen and routed to a dialyzer for cleansing. The cleansed blood is then returned to the blood vessel through the infusion lumen. When such a catheter is used for hemodialysis, it is generally inserted into the body through the interior jugular vein, the subclavian vein, or the femoral vein. In addition to hemodialysis, extracorporeal catheters can also be used for other procedures in which a fluid is removed from the body for treatment and later returned to the body.

A variety of hemodialysis catheters are available. Among the types of commercially available catheters are: 1) a dual lumen catheter having one lumen (e.g., the blood infusion lumen), that terminates distal to the other lumen (e.g., the blood withdrawal lumen). Some catheters of this type are provided with a midline split between the withdrawal and infusion lumens, while others do not have such a split (e.g., the COOK® DDS catheter); 2) a catheter having a slifted valve in the distal tip that acts as a pressure valve opening. This valve opens inwardly for blood aspiration, outwardly for blood infusion, and remains closed when not in use (e.g., the Groshong catheter); 3) polyester-cuffed central venous silicone catheters that are tunneled underneath the skin to reduce infection (e.g., Broviac, Leonard and Hickman catheters); 4) a dual lumen catheter having a tapered tip and two adjacent holes communicating with one lumen just proximal to the tip to assist with outflow, and two adjacent holes communicating with the other lumen (180 degrees removed) just proximal to the first set of holes to assist with inflow (e.g., the Mahurkar catheter); and 5) a dual lumen catheter having a diverting structure consisting of a shoulder that has a straight up distal face and a sloped proximal face to reduce access recirculation and raise pressure in the vicinity of the inlet aperture (U.S. Pat. No. 6,409,700).

Typically, dual lumen hemodialysis catheters have fixtures and related structure at the proximal end that are larger than the diameter of an introducer device through which the catheter is inserted into the vessel. As a result, splittable introducer sheaths, such as the PEEL-AWAY® introducers commercially available from Cook, Incorporated, of Bloomington, Ind., are often utilized for insertion of the catheter. Although such introducers are generally effective for such use, it would be desirable if the catheter insertion procedure could be simplified in a manner such that a separate introducer sheath would not be required. Eliminating the introducer device simplifies the procedure by omitting the sheath removal step that must otherwise be carried out by the physician, and also reduces the overall cost of the procedure. However, since many conventional hemodialysis catheters have stepped or otherwise non-tapered distal (e.g., entry) portions, these catheters are generally not amenable to non-traumatic insertion in the vessel without the use of a tapered introducer and/or dilator.

It would be desirable to provide a multi-lumen catheter for use in the extracorporeal transport of bodily fluids that is capable of insertion into a vessel in substantially non-traumatic fashion, and without the necessity of utilizing a separate introducer apparatus.

### BRIEF SUMMARY

The present invention addresses the shortcomings of the prior art. In one form thereof, the invention comprises a multi-lumen catheter for use in the extracorporeal treatment of bodily fluids. The catheter comprises a tubular catheter body, wherein a first septum extends along a length of the catheter body. The catheter body has a withdrawal port and an infusion port. First and second withdrawal lumens are disposed on one side of the septum for transport of fluids withdrawn from a body vessel through the withdrawal port to an extracorporeal treatment unit. An infusion lumen is disposed on another side of the septum for infusion of treated fluids from the extracorporeal treatment unit through the infusion port into the vessel. The catheter body has a second septum extending along a length of the first septum. A proximal portion of the second septum separates the first and second withdrawal lumens. A distal portion of the second septum extends distal to the withdrawal port, and tapers to a termination point along the first septum length.

In another form thereof, the present invention comprises a catheter for use in the extracorporeal treatment of bodily fluids. The catheter comprises a generally cylindrical catheter body having a proximal end and a distal end, wherein the distal end tapers to a distal tip portion. The generally cylindrical catheter body has a first septum extending along the length of the catheter body, and has a withdrawal port and an infusion port axially spaced along the catheter body length. The catheter body has first and second withdrawal lumens disposed on one side of the septum for transport of the body fluid withdrawn from a body vessel through the withdrawal port to an extracorporeal treatment unit, and an infusion lumen disposed on another side of the septum for infusion of treated fluid from the extracorporeal treatment unit through the infusion port into the vessel. The catheter body has a second septum extending along a length of the first septum. A proximal portion of the second septum separates the first and second withdrawal lumens, and a distal portion of the second septum extends distal to the withdrawal port and tapers to a termination point along the first septum length.

In yet another form thereof, the invention comprises a method for treating a body fluid. A catheter is provided for transporting the body fluid. The catheter comprises a generally cylindrical catheter body having a proximal end and a distal end, wherein the distal end tapers to a distal tip portion. The catheter body has a first septum extending along its length, and has a withdrawal port and an infusion port. The catheter body has first and second withdrawal lumens disposed on one side of the first septum for transporting body fluid withdrawn from a body vessel through the withdrawal port to an extracorporeal treatment unit, and an infusion lumen disposed on another side of the septum for infusion of treated fluid from the extracorporeal treatment unit through the infusion port into the vessel. The catheter body has a second septum extending along a length of the first septum. A proximal portion of the second septum separates the first and second withdrawal lumens, and a distal portion of the second septum extends distal to the withdrawal port and tapers to a termination point along the first septum length. The distal end of the catheter body is inserted into the vessel, and the body fluid to be treated is withdrawn from the vessel through the withdrawal port. The withdrawn fluid is transported through the withdrawal lumens to a treatment instrument. Following treatment in the treatment instrument, the fluid is transported from the treatment instrument through the infusion lumen, and infused into the body vessel through the infusion port.

### BRIEF DESCRIPTION OF THE DRAWINGS

FIG. 1 is a side elevational view of a prior art hemodialysis catheter assembly;

FIG. 2 is an enlarged distal end view of the prior art catheter assembly of FIG. 1;

FIG. 3 is a top view of the distal end portion of the prior art catheter assembly of FIG. 1;

FIG. 4 is a side elevational view of the distal end portion of another prior art hemodialysis catheter assembly;

FIG. 5 is an enlarged distal end view of the prior art catheter assembly of FIG. 4;

FIG. 6 is a top view of the distal end portion of the prior art catheter assembly of FIG. 4;

FIG. 7 is a side elevational view of the distal end portion of a multi-lumen catheter according to an embodiment of the present invention;

FIG. 8 is a distal end view of the catheter of FIG. 7;

FIG. 9 is a top view of portion of the catheter of FIG. 7;

FIG. 10 is a bottom view of the catheter of FIG. 7;

FIG. 11 is a transverse sectional view taken along line 11-11 of the catheter of FIG. 7;

FIG. 11A is a transverse sectional view of an alternate embodiment of a multi-lumen catheter, wherein the horizontal septum 30A has been repositioned with reference to the catheter of FIG. 11 to adjust the relative flow rates between the withdrawal lumens and the infusion lumen;

FIG. 12 is a longitudinal sectional view taken along line 12-12 of FIG. 9;

FIG. 13 is a side elevational view of the distal end portion of another embodiment of a multi-lumen catheter according to the present invention;

FIG. 14 is a distal end view of the catheter of FIG. 13;

FIG. 15 is a top view of portion of the catheter of FIG. 13; and

FIG. 16 is a bottom view of the catheter of FIG. 13.

### DETAILED DESCRIPTION OF THE DRAWINGS AND THE PRESENTLY PREFERRED EMBODIMENTS

For purposes of promoting an understanding of the present invention, reference will now be made to the embodiments illustrated in the drawings, and specific language will be used to describe the same. It is nevertheless to be understood that no limitation of the scope of the invention is thereby intended, the scope of the invention being indicated by the claims appended below and the equivalents thereof. The figures are not all drawn to the same scale to avoid obscuring the details of the finer structures. The following detailed description of the preferred embodiments will make clear the preferred arrangement, size relationships and manner of using the components shown herein.

The present invention is directed to a multi-lumen catheter for use in the transport of bodily fluids for treatment external of the body, referred to in the medical arts as “extracorporeal” treatment. The bodily fluids are transported from the body through one or more withdrawal lumens in the catheter, and are thereafter transported to an instrument for extracorporeal treatment. The treated fluids are then returned, or infused, to the body through an infusion lumen in the catheter.

In the following discussion, the terms “proximal” and “distal” will be used to describe the axial ends of the catheter, as well as the axial ends of various component features. The “proximal” end is used in conventional manner to refer to the end of the catheter (or component) that is closest to the operator during use of the assembly. The “distal” end is used in conventional manner to refer to the end of the catheter (or component) that is initially inserted into the patient, or that is closest to the patient.

Those skilled in the art will appreciate that the catheter described herein is suitable for multiple uses involving inflow and outflow of bodily fluids. However, the invention will be primarily described hereinafter with reference to one of its intended uses, namely as a hemodialysis catheter for use in the extracorporeal treatment of blood. The hemodialysis catheter enables blood inflow without disturbance, and blood return without hemolysis. In addition to hemodialysis, the catheter can be used for other extracorporeal fluid treatments in which a body fluid is withdrawn from the body, subjected to a treatment process, and thereafter returned to the body. Pheresis and hemofiltration are non-limiting examples of such additional procedures.

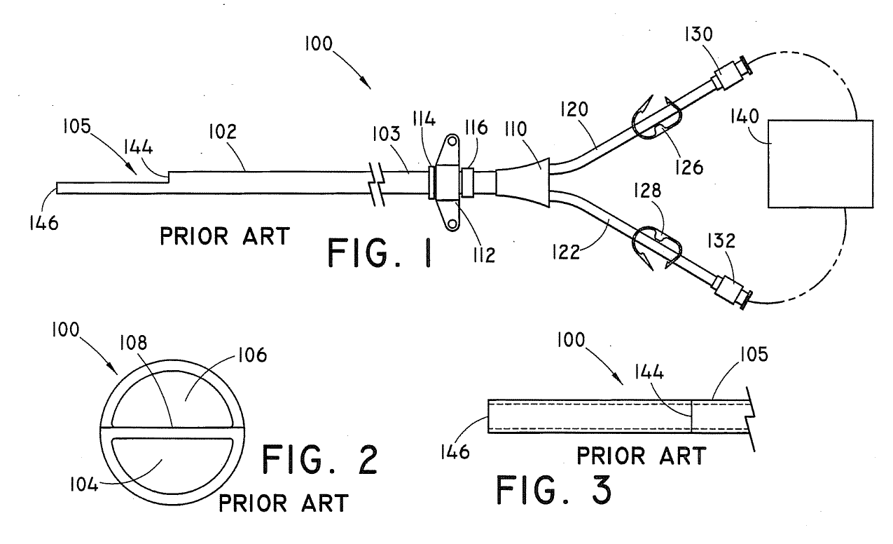

FIG. 1 is a side elevational view of a prior art hemodialysis catheter assembly 100. FIG. 2 is an enlarged end view taken from the distal end of prior art catheter assembly 100. FIG. 3 is a top view of the distal end portion of the catheter assembly 100. Prior art assembly 100 includes an elongated generally cylindrical catheter body 102 having a proximal end 103 and a distal end 105, and having dual lumens 104, 106 extending therethrough. Lumen 104 is separated from lumen 106 by septum 108.

Catheter assembly 100 includes a conventional bifurcated fitting, such as manifold 110, positioned at the proximal end of catheter body 102. Conventional suture wings 112 may be provided if desired. Stop mechanisms 114, 116 may be provided at each axial side of suture wings 112 to prevent catheter body 102 from axial movement relative to the suture wings. Flexible extension tubes 120, 122 extend in the proximal direction from manifold 110. Each extension tube is in fluid communication with a separate one of lumens 104, 106. Clamps 126, 128 are provided for selectively closing off fluid flow through the respective extension tubes 120, 122. Luer lock or other suitable connecting mechanisms 130, 132 are provided for engagement with a treatment instrument 140, such as a dialyzer, for establishing a flow path of blood to and from the dialyzer. Dialyzer 140 and its ingress and egress openings are shown schematically in FIG. 1.

In the prior art hemodialysis catheter assembly 100 shown in FIGS. 1-3, catheter body 102 includes a stepped axial surface along the length of catheter body distal end portion 105. Withdrawal port 144 communicates with lumen 106 for transporting fluid withdrawn from the body vessel through the catheter assembly to the dialyzer. Treated fluid returns from the dialyzer through lumen 104, and is returned to the vessel via infusion port 146. Typically, withdrawal port 144 is positioned proximal to infusion port 146. This arrangement reduces recirculation during hemodialysis. The arrangement also increases the efficiency of the procedure, since cleansed blood that has been returned to the vessel via infusion port 146 is not immediately withdrawn again and re-transported to the dialyzer for cleansing. Ports 144, 146 define a stepped arrangement, wherein port 144 is oriented substantially perpendicular to the vessel wall upon insertion of the apparatus. This orientation makes it difficult for the wall to collapse over the opening in the port and block the flow of blood.

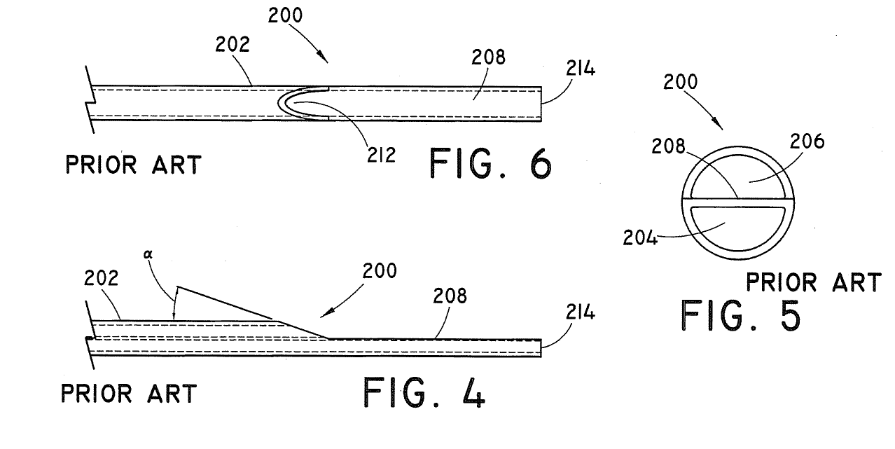

Another prior art hemodialysis catheter assembly 200 is shown in FIGS. 4-6. FIG. 4 is a side elevational view of the distal end portion of prior art hemodialysis catheter assembly 200. FIG. 5 is an enlarged end view taken from the distal end of the catheter assembly 200, and FIG. 6 is a top view of the portion of the prior art catheter assembly shown in FIG. 4. The proximal portion of assembly 200 is similar to that of prior art assembly 100.

Prior art assembly 200 includes an elongated generally cylindrical catheter body 202, and has dual lumens 204, 206 extending therethrough. Lumen 204 is separated from lumen 206 by septum 208. Withdrawal port 212 communicates with lumen 206 for transporting fluid withdrawn from the vessel through the catheter assembly to the dialyzer. Treated fluid returns to the vessel through lumen 204 and infusion port 214, in the same manner described with reference to the prior art embodiment of FIGS. 1-3.

Unlike the generally perpendicular orientation of the withdrawal port 144 of prior art assembly 100, the withdrawal port 212 of prior art assembly 200 comprises a straight angled cut. This design allows for easier insertion into the vessel when compared to assembly 100. However, the straight angled arrangement defined by angle a (FIG. 4) exposes a greater portion of the opening to the vessel wall, thereby increasing the possibility of blockage of the withdrawal port. On the other hand, the stepped arrangement of assembly 100 (FIGS. 1-3) minimizes the possibility of blockage. However, the assembly having the stepped arrangement cannot generally be inserted without the use of a removable sheath, such as the splittable sheaths discussed above.

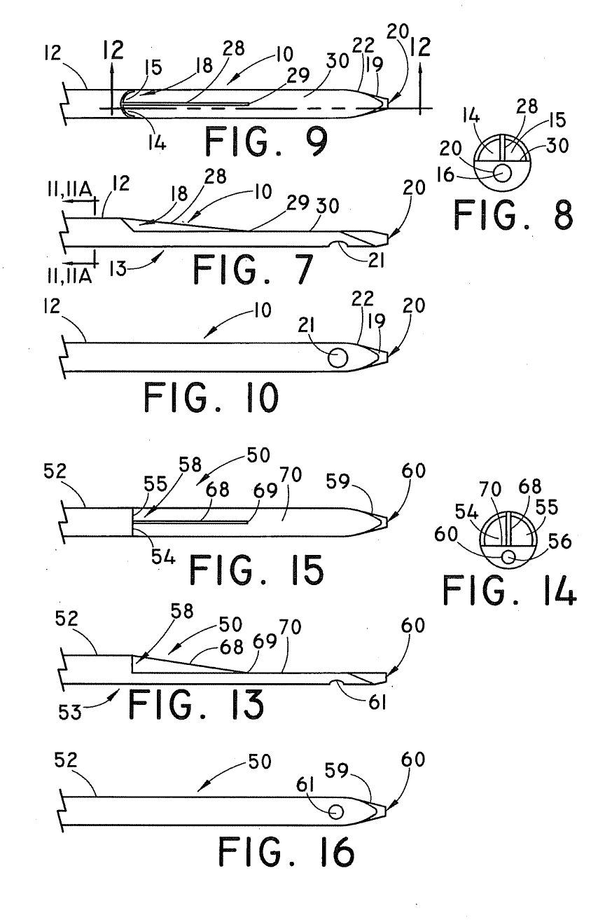

The multi-lumen catheter of the present invention may be readily understood by viewing an embodiment of the invention depicted in FIGS. 7-12. FIG. 7 is a side elevational view of the distal end portion 13 of a multi-lumen catheter 10 according to this embodiment. FIG. 8 is an end view of the catheter of FIG. 7. FIG. 9 is a top view of distal end portion 13 of the catheter of FIG. 7, and FIG. 10 is a bottom view of catheter distal end portion 13. FIG. 11 is a transverse sectional view taken along line 11-11 of the catheter of FIG. 7, and FIG. 12 is a longitudinal sectional view taken along line 12-12 of FIG. 9.

Multi-lumen catheter 10 includes an elongated tubular catheter body 12. Preferably, catheter body 12 has a generally cylindrical construction, and tapers to a distal tip portion 19. As best illustrated in the sectional view of FIG. 11, a first, or horizontal, septum 30 spans the length of catheter body 12. Septum 30 bisects the upper and lower portions of catheter body 12, thereby defining a surface that separates lumens 14, 15 from lumen 16. A second, or vertical septum 28 bisects portions of the upper catheter body, thereby defining a surface that separates lumens 14, 15. The terminology “upper”, “lower”, “horizontal”, and “vertical”, as utilized herein is based upon the orientation of the respective features when the catheter body 12 is positioned as shown in the figures. Those skilled in the art will appreciate that when in use, the catheter may be rotated, twisted, or otherwise maneuvered such that the respective features may not always be in the exact orientation shown in the figures. Nonetheless, for identification purposes in the discussion that follows, it is believed that these designations will assist those skilled in the art in understanding the structure of the inventive multi-lumen catheter 10.

In the preferred embodiment of FIGS. 7-12, upper lumens 14, 15 comprise respective withdrawal lumens, and lower lumen 16 comprises the infusion lumen. Preferably, the vertical septum 28 that separates withdrawal lumens 14, 15 extends to the proximal end of catheter body 12.

In use, the proximal end of catheter body 12 is preferably received in a conventional bifurcated fitting, such as manifold 110 shown in FIG. 1. When the catheter body proximal end is received in a bifurcated manifold, withdrawal lumens 14, 15 are received in one passageway of the bifurcated fitting, and infusion lumen 16 is received in the other passageway. Flexible extension tubes, such as tubes 120, 122, may be provided to extend in a proximal direction from the respective passageways in the bifurcated manifold in well-known manner. In this case, one extension tube will be in communication with withdrawal lumens 14, 15, whereas the other extension tube will be in communication with infusion lumen 16. Clamps 126, 128 for selectively closing off fluid flow through the respective extension tubes 120, 122, and Luer lock or other suitable connecting mechanisms 130, 132 for engagement with a treatment instrument 140, such as a dialyzer, may be provided as shown in FIG. 1. Conventional suture wings 112 and stop mechanisms 114, 116 may also be provided if desired.

As illustrated in FIGS. 7 and 9, a withdrawal port 18 communicates with withdrawal lumens 14, 15 for transporting fluid withdrawn from the vessel through the catheter assembly to the dialyzer (not shown) for treatment. Treated fluid returns to the vessel through infusion lumen 16. In the preferred embodiment shown, treated fluid through lumen 16 re-enters the vessel via both an infusion port 20 at the distal end of catheter body 12, and at least one optional side port 21 disposed along the length of the catheter body that communicates with the infusion lumen.

Catheter body 12 preferably includes a distal end portion 19 that tapers to infusion port 20 as shown in FIGS. 7-10. As best shown in FIG. 8, the cross-sectional area of the opening at infusion port 20 will typically be considerably smaller than the cross-sectional area of the remainder of the infusion lumen 16. As a result, the one or more side ports 21 are preferably provided to insure smooth fluid flow through the infusion lumen 16 and infusion port 20, and to inhibit fluid back-up in the infusion lumen.

Preferably, the total combined cross-sectional areas of infusion port 20 and side port(s) 21 is at least as great as the cross-sectional area of the infusion lumen. As a result, the infusion flow rate will not be reduced due to the reduction in diameter at the tapered distal tip, and fluid will not be backed-up in the infusion lumen. Preferably, side port 21 is spaced about 1 mm proximal to a transition point 22, designated as the point where the main catheter body portion 12 meets the tapered tip 19. Although a single side port 21 is illustrated in the embodiment shown herein, those skilled in the art will appreciate that additional side ports can be provided in the catheter body if desired. In order to minimize the possibility of re-mixing treated fluid passing through the side port(s) with untreated fluid entering the withdrawal port 18, it is preferred to maintain the side port(s) as close to the distal end of the catheter as practicable.

FIG. 11A illustrates an alternative embodiment of a catheter 10A. Catheter 10A is otherwise similar to the aforementioned catheter 10, but the horizontal septum 30A of catheter 10A has been repositioned with reference to horizontal septum 30. As a result, the relative cross sectional areas of the respective withdrawal lumens 14A, 15A, and the infusion lumen 16A has also been altered. In the example shown in FIG. 11A, the cross-sectional area of respective withdrawal lumens 14A, 15A has been increased relative to the cross-sectional area of infusion lumen 1 6A. By repositioning the horizontal septum, the relative flow rates through the respective withdrawal and infusion lumens can be increased, or decreased, as desired. Those skilled in the art will appreciate that the relative positioning of respective horizontal septums 30, 30A as shown in the figures is exemplary only, and that other positionings may be appropriate for a particular case. In a most preferred case, the septum will be positioned such that the flow rate through the withdrawal lumens will be the same as the flow rate through the infusion lumen and any side ports.

As stated, vertical septum 28 preferably extends to the proximal end of catheter body 12. If desired, however, the vertical septum 28 may terminate at any position within the catheter body prior to the proximal end. In this event, lumens 14, 15 would merge into a single withdrawal lumen from the point of termination of the septum to the proximal end of the catheter body. However, for ease of manufacturing, and to maintain optimal stiffness of catheter 10 at its proximal portion, it is generally preferred to extend vertical septum 28 to the proximal end of catheter body 12.

As best illustrated in FIGS. 7 and 9, vertical septum 28 tapers in the distal direction from withdrawal port 18 to a distal termination point 29 along the distal length of horizontal septum 30. The presence of the tapered vertical septum 28 minimizes any abrupt or traumatic edges of the catheter body that may be encountered by the body vessel as the distal end of the catheter body is inserted into the vessel. The presence of the tapered portion of septum 28 also minimizes the possibility that the withdrawal port will be obstructed by the vessel wall. By providing the catheter body with a tapered distal tip and an atraumatic vertical septum 28 as described, the catheter can be directly inserted into the vessel over a wire guide without the need of a splittable introducer sheath as described above.

Those skilled in the art will appreciate that the length and degree of inclination of the tapered septum 28 is generally not critical, as long as the septum has sufficient length and taper to provide the benefits described. However, in most instances, a longer taper is beneficial as it will provide less resistance during percutaneous insertion than a shorter taper. Similarly, the degree of taper of catheter body distal end 19 is generally not critical, as long as the angle of taper is sufficient to avoid undue trauma upon insertion of the catheter end into the vessel.

Another feature that may be varied to assist catheter insertion is the angle of the withdrawal port 18 relative to the horizontal septum 30. As illustrated in prior art FIGS. 4 and 6, and in FIGS. 7 and 9 of the preferred embodiment herein, the withdrawal port 18 may be structured at a gentle angle relative to the horizontal septum 30. Providing a gentle angle of the withdrawal port as shown also facilitates insertion of the catheter 10. This may be contrasted, for example, with the perpendicular angle shown at withdrawal port 144 in FIG. 1. Although the specific angle of inclination is generally not critical, care must be taken to avoid too extreme of an angle. When this occurs, a greater portion of the port opening is exposed to the vessel wall. This arrangement may increase the possibility of blockage of the withdrawal port when compared to a less extreme angle. On the other hand, as the angle approaches the perpendicular angle of the prior art catheter of FIG. 1, the port provide a small amount of resistance to smooth insertion of the catheter into the vessel. Additional discussion of this feature has been provided above with reference to FIGS. 4-6.

Thus, as described above, various features of the inventive catheter 10 may be modified as desired to facilitate insertion of the inventive catheter into a vessel in a manner that minimizes trauma to the patient. Those skilled in the art are believed capable of optimizing the variables described herein for a particular purpose, without requiring undue experimentation.

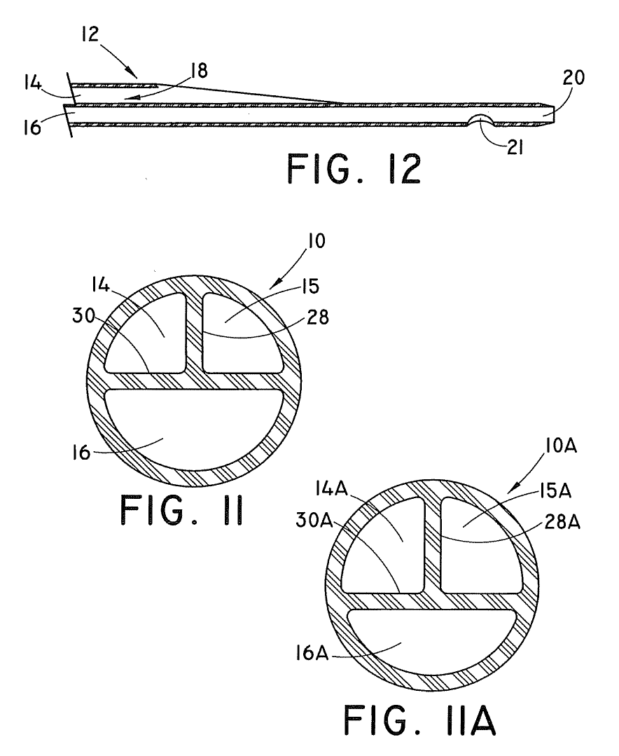

An alternative embodiment of a multi-lumen catheter 50 according to the invention is shown in FIGS. 13-16. Catheter 50 is similar to catheter 10 in the embodiment of FIGS. 7-12 in numerous respects. Multi-lumen catheter 50 includes an elongated generally cylindrical catheter body 52, only the distal portion 53 of which is visible in the figures. Catheter body 52 preferably tapers to a distal tip portion 59.

Once again, a horizontal septum 70 spans the length of catheter body 52, and bisects the upper and lower portions of catheter body 52. Vertical septum 68 bisects portions of the upper catheter body. Horizontal septum 70 defines a surface that separates withdrawal lumens 54, 55 from infusion lumen 56, and vertical septum 68 defines a surface that separates the two withdrawal lumens 54, 55. Withdrawal port 58 communicates with withdrawal lumens 54, 55 for transporting fluid withdrawn from the vessel to the dialyzer, and infusion port 60 and one or more side ports 61 communicate with infusion lumen 56 for transporting treated fluid back to the vessel.

The proximal portions of catheter 50 may be the same as the proximal portions of catheter 10. The distal portion of vertical septum 68 tapers in the distal direction from withdrawal port 58 to a distal termination point 69 along the distal length of horizontal septum 70 in the same manner as in the previous embodiment.

Unlike catheter 10, the withdrawal port 58 of catheter 50 is perpendicular to the horizontal septum 70, in the manner of prior art sheath 100. With this configuration, it is less likely that the withdrawal port will be occluded by the vessel wall, when compared to an angled withdrawal port. However, as stated, this configuration may be more likely to impede smooth insertion of the catheter into the vessel in some instances when compared to the angled withdrawal port 18.

Catheter body 12, 52 may be formed from a conventional polymer commonly used in the medical arts for such purposes, such as radiopaque polyurethane. Other conventional materials used for such purposes in the medical arts may be substituted. Non-limiting examples of such materials include silicone, polyurethane and PTFE.

Typically, catheter body 12, 52 will be formed by a conventional extrusion process. The exposed portions of vertical septum 28, 68 (i.e., the portions of the vertical septum distal to withdrawal ports 18, 58), can be formed by cutting away unnecessary distal portions of the extruded catheter body. Alternatively, the exposed portions of the vertical septum can be formed by a secondary molding process. This process would involve cutting away the upper portion of the extruded catheter body 12, 52 distal of withdrawal port 18, 58, and molding the exposed portions of vertical septum 28, 68 onto horizontal septum 30, 70 distal of the withdrawal port as shown. The one or more side ports may be formed in conventional fashion, such as by punching or skiving the ports through catheter body 12, 52.

Insertion of the catheter into the vessel can be made over a wire guide, e.g., via the well-known Seldinger percutaneous entry technique. Transport of bodily fluid to the dialyzer and return of the treated fluid to the body vessel follows a path substantially similar to that of the prior art embodiments previously described, and need not be further discussed for an understanding of the present invention.

It is therefore intended that the foregoing detailed description be regarded as illustrative rather than limiting, and that it be understood that it is the following claims, including all equivalents, that are intended to define the spirit and scope of this invention.

1. A multi-lumen catheter for use in the extracorporeal treatment of bodily fluids, comprising:
a tubular catheter body having a proximal end, a distal end, and a length, and having a first septum extending along said length, said catheter body having a withdrawal port and an infusion port, said ports being axially spaced along said catheter body length such that said withdrawal port is disposed proximal to said infusion port along said length, of said distal end tapering from a larger diameter to a smaller diameter distal tip portion, said catheter body having first and second withdrawal lumens disposed on one side of said septum for transport of fluids withdrawn from a body vessel through said withdrawal port to an extracorporeal treatment unit, and an infusion lumen disposed on another side of said septum for infusion of treated fluids from said extracorporeal treatment unit through said infusion port into the vessel, said catheter body having a second septum extending along a length of said first septum, a proximal portion of said second septum separating said first and second withdrawal lumens, and a distal portion of said second septum extending distal to said first and second withdrawal lumens and tapering to a termination point along said first septum length.

2. (canceled)

3. (canceled)

4. The catheter of claim 1, wherein said infusion port comprises an opening at the distal tip portion of said catheter body.

5. The catheter of claim 4, wherein said catheter body further comprises at least one side port communicating with said infusion lumen for infusion of treated fluids into the vessel.

6. The catheter of claim 5, wherein said infusion port and said side port each have a cross-sectional area, and wherein a combined cross-sectional area of said infusion port and said side port is at least as great as a cross-sectional area of said infusion lumen.

7. The catheter of claim 5, wherein said side port is disposed distal to said withdrawal port along said catheter body length.

8. The catheter of claim 1, wherein said withdrawal port is disposed along said catheter body at an inclined angle for facilitating entry of a distal end of said catheter into said vessel.

9. The catheter of claim 4, wherein said termination point is proximal to said tapered distal end of said catheter body.

10. A multi-lumen catheter for extracorporeal treatment of a bodily fluid, comprising:
a generally cylindrical catheter body having a proximal end and a distal end, said distal end tapering to a distal tip portion, said generally cylindrical catheter body having a length, and having a first septum extending along said length, said catheter body having a withdrawal port and an infusion port, said withdrawal port and said infusion port being axially spaced along said catheter body length, said withdrawal port being oriented generally perpendicular to said first septum, said catheter body having first and second withdrawal lumens disposed on one side of said first septum for transport of said body fluid withdrawn from a body vessel through said withdrawal port to an extracorporeal treatment unit, and an infusion lumen disposed on another side of said first septum for infusion of treated fluid from said extracorporeal treatment unit through said infusion port into the vessel, said catheter body having a second septum extending along a length of said first septum, a proximal portion of said second septum separating said first and second withdrawal lumens, and a distal portion of said second septum extending distal to said withdrawal first and second withdrawal lumens and tapering to a termination point along said first septum length, said second septum sized and positioned for minimizing resistance during insertion of said catheter into said vessel.

11. The catheter of claim 10, wherein said infusion port comprises an opening at the distal tip portion of said catheter body.

12. The catheter of claim 11, wherein said catheter body further comprises at least one side port communicating with said infusion lumen for infusion of treated fluids into the vessel.

13. The catheter of claim 12, wherein said infusion port and said side port each have a cross-sectional area, and wherein a combined cross-sectional area of said infusion port and said side port is at least as great as a cross-sectional area of said infusion lumen.

14. (canceled)

15. The catheter of claim 10, wherein said catheter body is formed from silicone, polyurethane or PTFE.

16. A method for treating a body fluid, comprising:
providing a catheter for transporting said body fluid, said catheter comprising a generally cylindrical catheter body having a proximal end and a distal end, said distal end tapering to a distal tip portion, said generally cylindrical catheter body having a length, and having a first septum extending along said length, said catheter body having a withdrawal port and an infusion port, said withdrawal port being proximal to said infusion port along said catheter body length, said catheter body having first and second withdrawal lumens disposed on one side of said septum for transport of said body fluid withdrawn from a body vessel through said withdrawal port to an extracorporeal treatment unit, and an infusion lumen disposed on another side of said septum for infusion of treated fluid from said extracorporeal treatment unit through said infusion port into the vessel, said catheter body having a second septum extending along a length of said first septum, a proximal portion of said second septum separating said first and second withdrawal lumens, and a distal portion of said second septum extending distal to said first and second withdrawal lumens and tapering to a termination point along said first septum length;
inserting said distal end of said catheter body into said vessel;
withdrawing body fluid to be treated from said vessel through said withdrawal port;
transporting said withdrawn fluid through said withdrawal lumens to a treatment instrument;
treating said fluid in said treatment instrument;
transporting said treated fluid from said treatment instrument through said infusion lumen; and
infusing treated fluid into said body vessel through said infusion port.

17. The method of claim 16, wherein said infusion port comprises an opening at the distal tip of said catheter body, and wherein said catheter body further includes at least one side port along the length of said catheter body in communication with said second lumen for infusion of treated fluid into said body vessel.

18. The method of claim 17, wherein each of said infusion port and said side port have a cross-sectional area, and wherein the combined cross- sectional areas of said infusion port and said side port is at least as great as a cross- sectional area of said infusion lumen.

19. (canceled)

20. The method of claim 18, wherein said treatment unit comprises a dialyzer.

21. The catheter of claim 1, wherein said termination point of said second septum is disposed along said first septum length between said withdrawal lumens and said distal tip portion, said second septum having a length and taper structured and arranged for minimizing resistance during insertion of said catheter into said vessel.

22. The catheter of claim 21, wherein said termination point is substantially midway between said withdrawal lumens and said distal end along said first septum length.
